(19) United States
(12) Patent Application Publication
(10) Pub. No.: US 2015/0075252 A1
(43) Pub. Date: Mar. 19, 2015

(54) Method Of Determining Wear Abrasion Resistance Of Polycrystalline Diamond Compact (PDC) Cutters

(71) Applicants: Federico Bellin, Tomball, TX (US); Vamsee Chintamaneni, Houston, TX (US)

(72) Inventors: Federico Bellin, Tomball, TX (US); Vamsee Chintamaneni, Houston, TX (US)

(73) Assignee: VAREL INTERNATIONAL IND., L.P., Carrollton, TX (US)

(21) Appl. No.: 14/027,874

(22) Filed: Sep. 16, 2013

(51) Int. Cl. G01N 3/56; G01N 33/40

(52) U.S. Cl. CPC G01N 3/56; G01N 33/40
USPC 73/7

(57) ABSTRACT

The present disclosure provides methods and techniques for determining wear abrasion resistance of superhard components, such as cutters used in down-hole drilling tools. The methods and techniques provided herein produce an efficiency ratio of a superhard component through data obtained from a vertical turret lathe test. The efficiency ratio is the ratio between the volume of a target cylinder removed by the superhard component during the vertical turret lathe test and the normal force applied onto the superhard component by the target cylinder. The efficiency ratio is indicative of the energy efficiency of the superhard component.

### TECHNICAL FIELD

The present invention relates generally to methods for testing PDC cutters or other superhard components; and more particularly, to methods for testing and evaluating the abrasive wear resistance of PDC cutters or other superhard components.

### BACKGROUND

Down-hole tools used in drilling operations typically include a superhard component, such as a cutter inserted with the down-hole tool. The superhard component cuts or grinds away rock bits to create path in a rock formation for the remainder of the tool or tool string. The superhard component typically includes a cutting table fabricated from a superhard material such as polycrystalline diamond (“PCD”) or polycrystalline cubic boron nitride (“PCBN”). Common problems associated with these superhard components include chipping, spalling, partial fracturing, cracking, flaking, or dulling of the cutting table. These problems can result in the early failure of the cutting table. Typically, high magnitude stresses generated on the cutting table at the region where the cutting table makes contact with earthen formations during drilling can cause these problems. Such problems increase the cost of drilling due to costs associated with repair, production downtime, and labor costs. For these reasons, testing methods have been developed to ascertain the abrasion resistance and/or impact resistance of superhard components so that improved cutter longevity is achieved and the problems mentioned above are substantially reduced.

Superhard components, which include PDC cutters, have been tested for abrasive wear resistance through the use of two conventional testing methods. Early in the development of PDC materials, the abrasive wear resistance was tested using a conventional granite log test. However, as the PDC cutters became more wear resistant, and too much time and conventional target cylinders were required to complete the conventional granite log test, the conventional vertical turret lathe test (“VTL”) test replaced the conventional granite log test for testing abrasive wear resistance. Both the conventional granite log test and the vertical turret lathe test involved relative motion between the cutter and a target material, in which the cutter cuts away at the target material as it moves across a surface of the target material. In both the conventional vertical turret lathe test and the conventional granite log test, wear resistance of a cutter is determined as a ratio between the amount of target material removed and the amount of the cutting table of the cutter removed. This is known as a G-ratio and is commonly used to measure and compare cutter performance. However, the G-ratio is purely a geometric parameter and does not take into account the energetic efficiency of the cutting process. Specifically, two cutters with the same G-ratio may require very different cutting forces depending on the sharpness of the cutting table. Thus, these two cutters may require difference amounts of energy and thus have different effective lifetimes down-hole. However, the G-ratio is not indicative of this.

### SUMMARY

According to an example embodiment of the present disclosure, a method of testing a superhard component includes obtaining a superhard material, obtaining a target material having a volume and a surface, and contacting the superhard material to the surface of the target material. The method further includes removing a portion of the target material with the superhard material by moving the superhard material along the surface of the target material with reference to the target material. The method measures a normal force applied to the superhard material by the surface of the target material, and determines a ratio between the normal force and the value of a first variable.

According to another aspect of the present disclosure, a method of determining relative wear resistance of superhard components includes obtaining a first data set from a first vertical turret lathe test of a first superhard material, in which the first data set comprises a first normal force and a first variable. The method also includes obtaining a second data set from a second vertical turret lathe test of a second superhard material, in which the second data set comprises a second normal force and a second variable. The method then determines a first ratio of the value of the first variable to the first normal force and also determines a second ratio of the value of the second variable to the second normal force.

According to yet another example embodiment of the present disclosure, a method of determining relative wear resistance of superhard components includes obtaining a first efficiency ratio of a first superhard material. The first efficiency ratio includes a ratio of a first amount of a first target material removed to a first normal force applied to the first superhard material by the first target material under a first set of test conditions. The method further obtains a second efficiency ratio of a second superhard material. The second efficiency ratio comprises a ratio of a second amount of a second target material removed to a second normal force applied to the second superhard material by the second target material under a second set of test conditions. The first wear resistance value is then compared to the second wear resistance value.

### BRIEF DESCRIPTION OF THE DRAWINGS

The foregoing and other features and aspects of the invention are best understood with reference to the following description of certain exemplary embodiments, when read in conjunction with the accompanying drawings, wherein:

FIG. 1 is a perspective view of a superhard component that is insertable within a downhole tool in accordance with an exemplary embodiment of the present disclosure;

FIG. 2 is a side view of a vertical turret lathe test for testing abrasive wear resistance of a superhard component, in accordance with an exemplary embodiment of the present disclosure;

FIG. 3 is a flowchart of a method for testing the abrasive wear resistance of a superhard component, in accordance with exemplary embodiments of the present disclosure;

FIG. 4 is a flowchart of a method for ranking the abrasive wear resistance of multiple superhard components, in accordance with exemplary embodiments of the present disclosure; and

FIG. 5 is a graph of efficiency ratios for a plurality of superhard components, in accordance to exemplary embodiments of the present disclosure.

The drawings illustrate only exemplary embodiments of the invention and are therefore not to be considered limiting of its scope, as the invention may admit to other equally effective embodiments.

### BRIEF DESCRIPTION OF EXEMPLARY EMBODIMENTS

The present invention is directed to a method and apparatus for testing the abrasive wear resistance of superhard components. Although the description of exemplary embodiments is provided below in conjunction with a PDC cutter, alternate embodiments of the invention may be applicable to other types of superhard components including, but not limited to, PCBN cutters or other superhard components known or not yet known to persons having ordinary skill in the art. Additionally, though the following description of example embodiments makes reference to vertical turret lathe testing techniques, alternative embodiments of the invention are used with other cutter testing techniques, include granite log testing techniques.

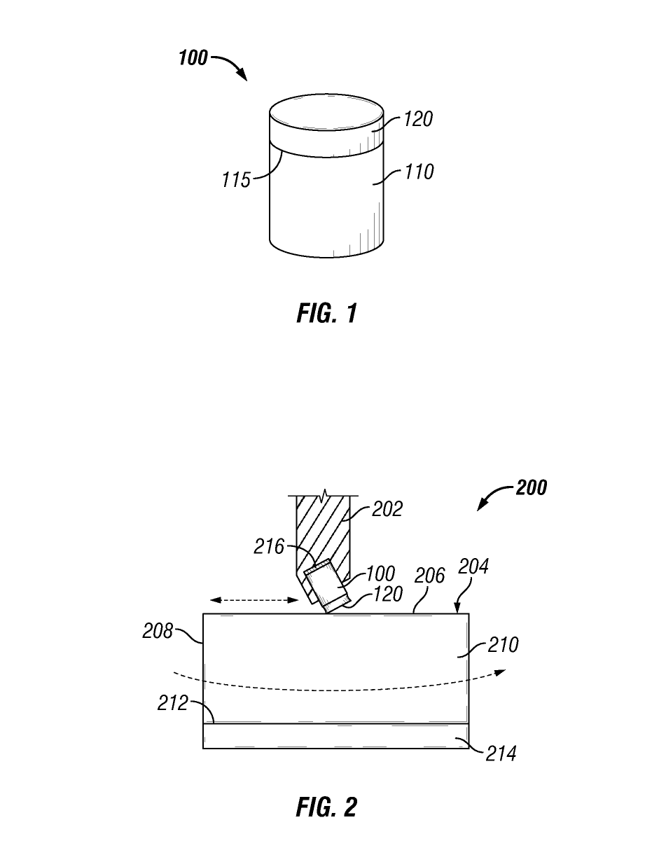

The invention is better understood by reading the following description of non-limiting, exemplary embodiments with reference to the attached drawings, wherein like parts of each of the figures are identified by like reference characters, and which are briefly described as follows. FIG. 1 is a perspective view of a superhard component 100 that is insertable within a downhole tool (not shown) in accordance with an exemplary embodiment of the invention. One example of a superhard component 100 is a cutting element 100, or cutter, for rock bits. The cutting element 100 typically includes a substrate 110 having a contact face 115 and a cutting table 120. The cutting table 120 is fabricated using an ultra hard layer which is bonded to the contact face 115 by a sintering process. The substrate 110 is generally made from tungsten carbide-cobalt, or tungsten carbide, while the cutting table 120 is formed using a polycrystalline ultra hard material layer, such as polycrystalline diamond (“PCD”), polycrystalline cubic boron nitride (“PCBN”), or tungsten carbide mixed with diamond crystals (impregnated segments). These cutting elements 100 are fabricated according to processes and materials known to persons having ordinary skill in the art. The cutting element 100 is referred to as a polycrystalline diamond compact (“PDC”) cutter when PCD is used to form the cutting table 120. PDC cutters are known for their toughness and durability, which allow them to be an effective cutting insert in demanding applications. Although one type of superhard component 100 has been described, other types of superhard components 100 can be utilized.

FIG. 2 is a side view of a vertical turret lathe test 200 for testing abrasive wear resistance of a superhard component 100, in accordance with embodiments of the present disclosure. Although one exemplary apparatus configuration for the vertical turret lathe 200 is provided, other apparatus configurations can be used without departing from the scope and spirit of the exemplary embodiment. The vertical turret lathe test 200 includes a rotating table 214 and a tool holder 202 positioned above the rotating table 214. The vertical turret lathe is used with a target cylinder 210 which is disposed atop the rotating table 214. The target cylinder 210 has a first end 212, a second end 204, and a sidewall 208 extending from the first end 212 to the second end 204. According to certain example embodiments, the second end 204 includes an exposed surface 206 which makes contact with a superhard component's cutting table 120 during the test. In an example embodiment, the target cylinder 210 is typically about thirty inches to about sixty inches in diameter, but can be smaller or larger depending upon the testing requirements.

The first end 212 of the target cylinder 210 is mounted on the lower rotating table 214 of the vertical turret lathe 200, and the exposed surface 206 faces the tool holder 202. The PDC cutter 100 is mounted in the tool holder 202 above the target cylinder's exposed surface 206 and makes contact with the exposed surface 206. The target cylinder 210 is rotated via the rotating table 214 as the tool holder 202 cycles the PDC cutter 100 from the center of the conventional target cylinder's exposed surface 206 out to its edge and back again to the center of the conventional target cylinder's exposed surface 206, or along its radius. In certain alternate embodiments, the tool holder 202 and PDC cutter 100 are stationary and the target cylinder moves laterally back and forth, with or without rotation. Thus, motion of the PDC cutter 100 on the target cylinder 210 refers to a relative motion between the PDC cutter 100 and the target cylinder 210. As the PDC cutter 100 contacts and moves across or along the exposed surface 206 of the target cylinder 210, the PDC cutter 100 removes, or cuts away, a portion of the target cylinder 210. In certain example embodiments, the tool holder 202 has a predetermined depth of cut. Thus, the volume of target cylinder 210 that is removed has a constant relationship to the distance of travel between the PDC cutter 100 and the target cylinder 210.

The vertical turret lathe further includes a load cell 216 disposed within the tool holder 202 between the tool holder 202 and the PDC cutter 100. The load cell 216 senses one or more components of force applied to the PDC cutter 100 from the target cylinder 210. In an example embodiment, the load cell 216 senses a normal force applied to the PDC cutter 100 by the target cylinder 210, the normal force being perpendicular to the exposed surface 206 of the target cylinder 210. In another example embodiment, the load cell 216 senses the normal force as well as two other components of force applied to the PDC cutter 100 by the target cylinder 210, in which the two other components of force are perpendicular to each other as well as to the normal force. Specifically, in certain example embodiments, the two other components of force include a tangential force, which is a force coming into the PDC cutter 100 from the spinning motion of the target cylinder 210, and a radial force, which is a force generated by the resistance against the PDC cutter 100 as the PDC cutter 100 traverses the radius of the target cylinder 210. The load cell 216 feeds the collected force data to a computer or other data processor where it can be observed or analyzed. In an example embodiment, the load cell 216 handles data acquisition at 7 kHz, delivering 7000 data points per second for each component of force. However, such high data resolution may be noisy and very data heavy, making the data more difficult to handle and process. Thus, various data averaging or sampling techniques may be utilized to provide a more usable data set. The data can be averaged or sampled to provide a lower data resolution. For example, in an exemplary embodiment, three data points are collected per pass of the PDC cutter 100 across the radius of the target cylinder 210, in which the three data points can be averages of multiple data points or samples.

In certain example embodiments, the target cylinder 210 is fabricated entirely from granite; however, the target cylinder 210 can be fabricated entirely from another single uniform natural or manmade material that includes, but is not limited to, Jackfork sandstone, Indiana limestone, Berea sandstone, Carthage marble, Champlain black marble, Berkley granite, Sierra white granite, Texas pink granite, Georgia gray granite, concrete, and the like. Additionally, the target cylinder 210 can be fabricated from two or more different materials. The target cylinder 210 has a compressive strength of about 25,000 psi or less and an abrasiveness of about 6 CAI or less when natural rock types are used. The conventional cylinder 210 has a compressive strength of about 12,000 psi or less and an abrasiveness of about 2 CAI or less when concrete is used. These example compressive strength values and CAI values are provided herein as a guidance, and the values may differ in other example embodiments. The abrasive wear resistance for the PDC cutter 100 is determined as an efficiency ratio, which is defined as the volume of target cylinder 210 that is removed by the PDC cutter 100 before or when the normal force on the PDC cutter 100, as measured by the load cell 216, reaches a certain normal force threshold to the value of the normal force threshold. Alternatively, instead of using volume of rock removed, the total distance that the PDC cutter 100 travels across the conventional target cylinder 210 or the duration of test before the normal force threshold is reached can also be measured and used to quantify the abrasive wear resistance or efficiency ratio for the PDC cutter 100. Such alternate data parameters are useful because they can be linearly manipulated to obtain the volume of rock removed, and thus, have a direct correlation to the volume of rock removed.

In performing a vertical turret lathe test, a number of testing conditions are defined, such parameters including depth of cut, feed rate, rake angles, rotation speed of the target cylinder, type and shape of the target cylinder, and other milling conditions such as moisture levels, temperature, and the like. The vertical turret lathe test can be used to compare or rank several distinct cutters 100 to each other. The vertical turret lathe test can also be used to compare or rank the performance or efficiency of the same cutter 100 under a range of testing conditions, such as those listed above.

In comparing several distinct cutters 100, all of the testing conditions are held constant, such that resulting differences in efficiency ratios between the cutters 100 is attributable to the distinct cutters 100. Inversely, in comparing the performance of the same cutter 100 under different testing conditions, the physical properties of the cutters 100 tested are held constant and one (or more) testing parameter is varied. Thus, the difference in the efficiency ratio between tests is attributable to the testing parameter that is varied. For example, the same type of cutter 100 may be tested at varying back rake angles. Thus, the resulting efficiency ratios of each test can be ranked to show which back rake angle is most advantageous under the other given test conditions for the specific type of cutter 100 tested.

In certain exemplary embodiments, the efficiency ratios of the tested cutters 100 (distinct or identical), are defined at the same normal force, or the normal force threshold. The normal force threshold is chosen to be appropriate for the type of target cylinder 210 used as well as the known performance capabilities of the tested cutters 100. With regard to the type of target cylinder 210 used, typically the harder the material of the target cylinder, the higher the normal force threshold is, because higher normal force is required for the overall test. For example, vertical turret lathe tests using concrete may have a normal force threshold of 500 lbs., while vertical turret lathe tests using granite, which is much harder than concrete, may have a normal force threshold of 6000 lbs. In certain cases, when testing a new cutter 100 on a new target cylinder 210, preliminary test runs are performed to determine an appropriate normal force threshold for the cutter/target cylinder combination.

Choosing the appropriate normal force threshold or a series of VTL tests of distinct cutters 100 may also include known performance capabilities of the tested cutters 100. Specifically, in certain example embodiments, the normal force threshold is chosen to be high enough such that all the tested cutters 100 in a comparative group are capable of reaching the normal force threshold.

In certain example embodiments, the vertical turret lathe test 200 is stopped when the normal force threshold is reached, and the volume of target cylinder 210 removed during the test is determined. This indicates how much energy it took for the specific cutter 100 to remove the volume of target cylinder 210. In actual drilling applications, more and more force is applied to the cutters 100 to keep drilling at a constant speed. When it is not appropriate to apply any more force or when the load of drilling is too high, drilling is stopped. Thus, the vertical turret lathe test 200 and the resultant efficiency ratio is indicative of which type of cutter or which settings will allow for longer and more efficient drilling, as a higher efficiency ratio may be directly correlated to the cutter staying sharper for a longer time, thus requiring less force.

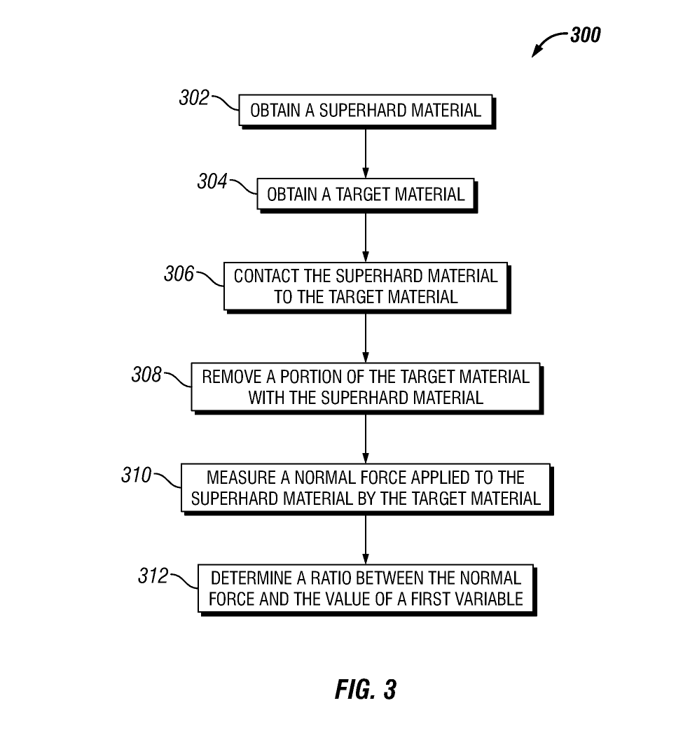

FIG. 3 illustrates a method 300 for testing a superhard component, such as a PDC cutter 100 (FIGS. 1 and 2), in accordance with embodiments of the present disclosure. Referring to FIG. 3, the method 300 includes obtaining a superhard material at step 302. In certain example embodiments, the superhard material is a PDC cutter 100 (FIG. 1) or other type of cutter 100 (FIG. 1). The method 300 further includes obtaining a target material at step 304. In certain example embodiments, the target material is a target cylinder 210 (FIG. 2) as described above and can be fabricated from any of the aforementioned materials and compositions. The method 300 includes contacting the superhard material to the target material at step 306 and removing a portion of the target material with the superhard material at step 308. In certain example embodiments, in removing a portion of the target material, the superhard material travels across a surface, such as the exposed surface 206 (FIG. 2), of the target material 210 (FIG. 2) with a specific depth of cut. In an exemplary embodiment, traveling across the surface of the target material includes moving back and forth between the center or inner radius of the target material to the outer radius of the target material if the target material is cylindrically shaped. In alternative embodiments, the superhard material moves in alternate paths about the surface of the target material. In an example embodiment, the superhard material 100 (FIG. 1) removes the portion of the target material at a constant depth of cut. In establishing a depth of cut of the superhard material onto the target material, a normal force is created and applied onto the superhard material from the target material, the normal force being perpendicular to the surface of the target material. Thus, the method further includes measuring the normal force applied onto the superhard material from the target material at step 310. In an exemplary embodiment, the normal force is collected by the load cell 216 (FIG. 2). In certain example embodiments, the load cell 216 also measures other components of force applied onto the superhard material by the target material. Finally, in certain example embodiments, the method 300 includes determining a ratio between the value of a variable related to the test and the normal force, in which the normal force and the value are correlated by time at step 312. For example, the ratio is between the value of the variable at the time the normal force reaches a predetermined threshold and the value of the normal force threshold. In an exemplary embodiment, the variable is the volume of the target material removed by the superhard material in the time it took for the normal force to reach the threshold. However, in other example embodiments, the variable can be another variable indicative of the volume of the target material removed, such as total distance traveled by the superhard material in relation to the target material, number of passes made by the superhard material, time, or the like. In certain example embodiments, given that certain parameters, such as depth of cut, feed rate, distance per pass, rotation speed of the target material, and the like, are held constant during the test, the total volume of target material removed can be calculated from one or more of these parameters. For example, given a known and constant depth of cut and rotation speed, the first variable can be the total distance travelled by the superhard component. The value of the total distance travelled, in conjunction with the values of the depth of cut and rotation speed, can be used to derive the volume of the target material removed.

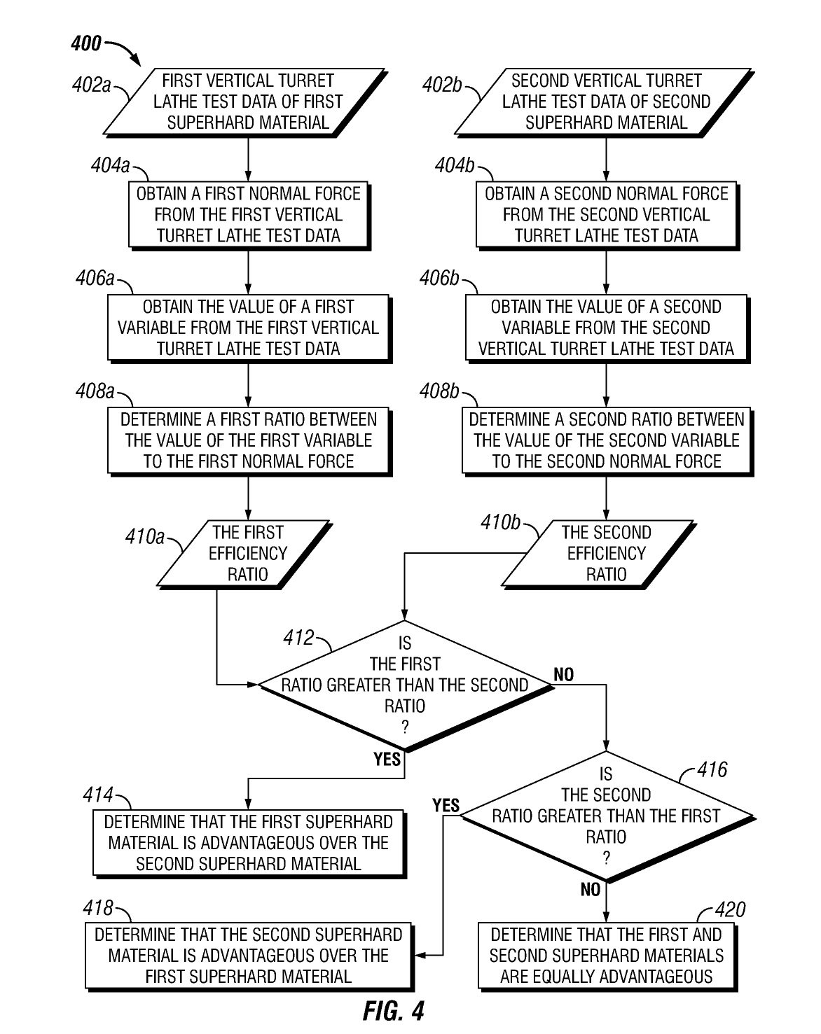

FIG. 4 illustrates a method 400 for determining relative wear resistance between one or more superhard materials, such as PDC cutters 100 (FIGS. 1 and 2), in accordance with example embodiments of the present disclosure. The method 400 includes using a first set of vertical turret lathe test data of a first superhard component at step 402a, and obtaining a first normal force from the first set of vertical turret lathe test data 402a at step 404a. The method 400 further includes obtaining the value of a first variable from the first set of vertical turret lathe test data 402a at step 406a. The value of the first variable is correlated with the value of the first normal force by time. In an exemplary embodiment, the obtained value of the first variable is the value of the first variable when the first normal force reaches a predetermined threshold, the threshold being the value of the first normal force obtained in step 404a. The first variable is a volume of a target material removed by the first superhard material during the first vertical turret lathe test according to some exemplary embodiments. In alternative embodiments, the first variable is another parameter through which the volume of target material removed can be calculated. The method 400 further includes determining a ratio between the value of the first variable and the value of the first normal force at step 408a, thereby providing the first efficiency ratio at step 410a.

Additionally, the method 400 further includes using a second set of vertical turret lathe test data of a second superhard component at step 402b, and obtaining a second normal force from the second set of vertical turret lathe test data 402b at step 404b. The method 400 further includes obtaining the value of a second variable from the second set of vertical turret lathe test data 402b at step 406b. The value of the second variable is correlated with the value of the second normal force by time. In an exemplary embodiment, the obtained value of the second variable is the value of the second variable when the second normal force reaches a predetermined threshold, the threshold being the value of the second normal force obtained in step 404b. The second variable is a volume of a target material removed by the second superhard material during the second vertical turret lathe test. In alternative embodiments, the second variable is another parameter through which the volume of target material removed can be calculated. The method 400 further includes determining a second ratio between the value of the second variable and the value of the second normal force at step 408b, thereby providing the second efficiency ratio at step 410b.

In certain example embodiments, the method 400 further includes comparing the first efficiency ratio from step 410a and the second efficiency ratio from step 410b and determining if the first efficiency ratio from step 410a is greater than the second efficiency ratio from step 410b at step 412. If the first efficiency from step 410a is indeed greater than the second efficiency ratio from step 410b, then the method 400 determines that the first superhard material is advantageous over the second superhard material at step 414. Specifically, this indicates that the first superhard material is advantageous over the second superhard material under the respective testing conditions of the first and second vertical turret lathe tests. Likewise, if the first efficiency ratio is not greater than the second efficiency ratio, then the method 400 decides if the second efficiency ratio from step 410b is greater than the first ratio from step 410a at step 416. If the second efficiency ratio is greater than the first efficiency ratio, then the method 400 determines that the second superhard material is advantageous over the first superhard material at step 418. If the second efficiency ratio is not greater than the first efficiency ratio, meaning the first and second efficiency ratios are the same, then the method 400 determines that the first and second superhard materials are equally advantageous at step 420. In certain exemplary embodiments, such conclusions are based on the first and second vertical turret lathe tests being performed under the same testing conditions, meaning the comparative advantage of the first and second superhard materials is applicable under the specific set of testing condition values. However, the results may or may not be true under another set of testing condition values.

In some example embodiments, the first and second superhard materials are distinct components, possibly having distinct physical or chemical compositions, distinct shapes, or another variant. In such embodiments, the first and second vertical turret lathe tests are performed under the same testing conditions (e.g., depth of cut, composition and size of target material, feed rate, rake angle, rotation speed, and the like) such that the only difference between the first and second vertical turret lathe tests is the superhard material used. Thus, difference in results, or efficiency ratio, can be attributed to the superhard materials used. Thus, the same vertical turret lathe test can be performed on several superhard materials to establish an efficiency ranking of the superhard materials.

In another example embodiment, the first and second superhard materials are substantially identical components, having substantially identical physical and chemical compositions and shapes. In such embodiments, at least one testing condition is varied between the first and second vertical turret lathe tests. Thus, difference in results, or efficiency ratios, between the first and second superhard materials can be attributed to the at least one testing condition that was varied. Thus, the same superhard material can be tested in vertical turret lathe tests having a varying parameter to rank performance of the superhard material under different values of the varying parameter. This allows the different values of said parameter to be ranked for effectiveness.

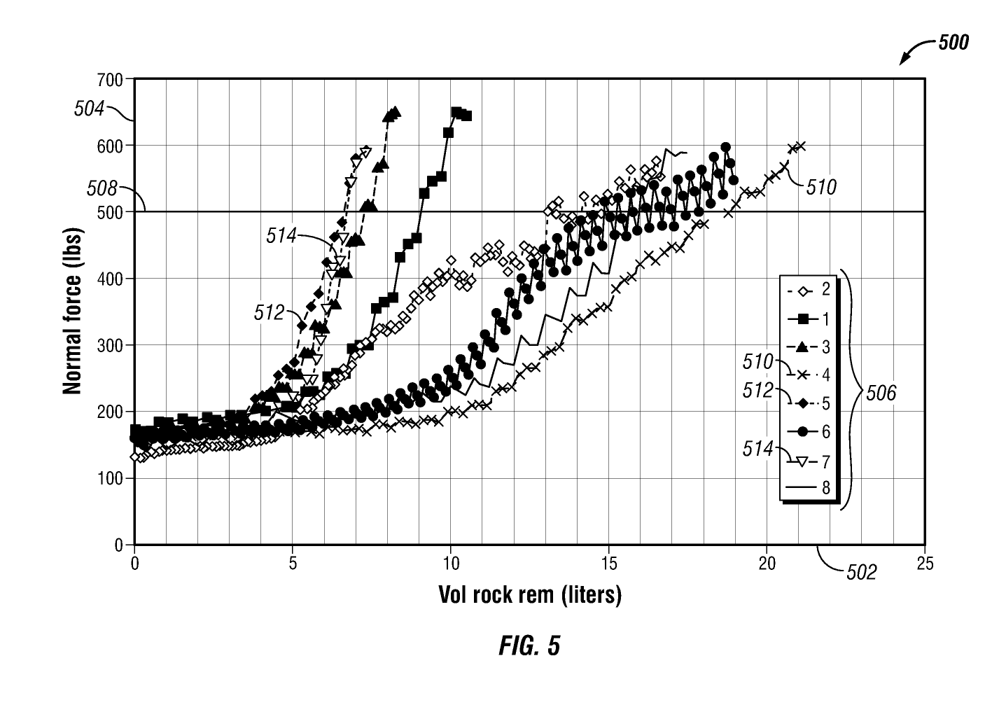

FIG. 5 illustrates a graph 500 of the efficiency ratios for eight superhard materials 506, similar to superhard material 100 (FIG. 1), in accordance with an example embodiment of the present disclosure. Referring to FIG. 5, the graph 500 is defined by volume of target material removed 502 as one axis, and the value of normal force 504 as another axis. Thus, any point on the graph 500 corresponds to a certain ratio of volume removed 502 to normal force 504, also known as an efficiency ratio. Accordingly, the higher the volume removed 502 and/or lower the normal force 504, the higher the efficiency. Thus, given the same normal force 504, the superhard material 506 with the highest volume removed 502 is the most efficient under the specific testing conditions and at the define normal force, as described above. For example, according to the graph 500, given a normal force 504 of 500 lbs., the 4^{th} superhard material 510 removed the highest volume 502 of the target material, while the 5^{th} superhard material 512 and 7^{th} superhard material 514 removed the lowest volume 502 of the target material, with the remaining superhard materials falling somewhere in between. These example results indicate that the 4^{th} superhard material 510 is the most efficient superhard material of the eight superhard materials 506 tested under the testing conditions and at 500 lbs 508 of normal force 504.

The efficiency ratio, which is indicative of the relationship between amount of rock removed and the downward normal force required by the cutter, allows cutters to be tested and evaluated with regard to energy efficiency. Thus, the methods and techniques disclosed herein provide a more realistic indication of cutter performance and usability under real field conditions.

Although each exemplary embodiment has been described in detail, it is to be construed that any features and modifications that are applicable to one embodiment are also applicable to the other embodiments. Furthermore, although the invention has been described with reference to specific embodiments, these descriptions are not meant to be construed in a limiting sense. Various modifications of the disclosed embodiments, as well as alternative embodiments of the invention will become apparent to persons of ordinary skill in the art upon reference to the description of the exemplary embodiments. It should be appreciated by those of ordinary skill in the art that the conception and the specific embodiments disclosed may be readily utilized as a basis for modifying or designing other structures or methods for carrying out the same purposes of the invention. It should also be realized by those of ordinary skill in the art that such equivalent constructions do not depart from the spirit and scope of the invention as set forth in the appended claims. It is therefore, contemplated that the claims will cover any such modifications or embodiments that fall within the scope of the invention.

What is claimed is:

1. A method of testing a superhard component, comprising:
obtaining a superhard material;
obtaining a target material comprising a volume and a surface;
contacting the superhard material to the surface of the target material;
removing a portion of the target material with the superhard material by moving the superhard material along the surface of the target material with reference to the target material;
measuring a normal force applied to the superhard material by the surface of the target material; and
determining a ratio between the normal force and the value of a first variable, wherein the first variable is indicative of a volume of target material removed.

2. The method of claim 1, wherein the normal force is measured by a load cell, wherein the load cell continuously collects normal force data as the superhard material moves along the surface of the target material.

3. The method of claim 1, wherein the variable comprises one of a distance of motion between the superhard material and the target material, an amount of time that the superhard material moves along the surface of the target material, a number of passes made by the superhard material across the surface of the target material, or any mathematical combination thereof.

4. The method of claim 1, wherein the superhard material comprises polycrystalline diamond.

5. The method of claim 1, wherein the target material comprises concrete, rock, a naturally occurring material, a synthetic material, or any combination thereof

6. The method of claim 2, further comprising:
averaging or sampling a plurality of values from the normal force data collected by the load cell.

7. The method of claim 1, further comprising:
removing the portion of the target material by the superhard material until the measured normal force reaches or exceeds a threshold force.

8. A method of determining relative wear resistance of superhard components, comprising:
obtaining a first data set from a first vertical turret lathe test of a first superhard material, wherein the first data set comprises a first normal force and a first variable;
obtaining a second data set from a second vertical turret lathe test of a second superhard material, wherein the second data set comprises a second normal force and a second variable;
determining a first ratio of the value of the first variable to the first normal force; and
determining a second ratio of the value of the second variable to the second normal force.

9. The method of claim 8, further comprising:
comparing the first ratio to the second ratio; and
determining that the first superhard material has greater wear resistance than the second superhard material when the first ratio is higher than the second ratio.

10. The method of claim 8, wherein the first normal force and second normal force have the same value.

11. The method of claim 10, further comprising:
ranking the first and second superhard components based on the values of the first and second variables.

12. The method of claim 8, wherein the first variable is indicative of a first amount of target material removed by the first superhard material in the first vertical turret lathe test, and the second variable is indicative of a first amount of target material removed by the second superhard material in the second vertical turret lathe test.

13. The method of claim 8, wherein the first vertical turret lathe test and the second vertical turret lathe test comprise a substantially identical set of testing conditions.

14. The method of claim 13, wherein the set of testing conditions comprises rake angle, feed rate, depth of cut, size and composition of target material, rotation speed of target material, or any combination thereof

15. The method of claim 8, wherein the first variable is a first amount of target material removed by the first superhard component when the first normal force is at a threshold value, and wherein the second variable is a second amount of target material removed by the second superhard component when the second normal force is at the threshold value.

16. A method of determining relative wear resistance of superhard components, comprising:
obtaining a first efficiency ratio of a first superhard material, wherein the first efficiency ratio comprises a ratio of a first amount of a first target material removed to a first normal force applied to the first superhard material by the first target material under a first set of test conditions;
obtaining a second efficiency ratio of a second superhard material, wherein the second efficiency ratio comprises a ratio of a second amount of a second target material removed to a second normal force applied to the second superhard material by the second target material under a second set of test conditions; and
comparing the first wear resistance value to the second wear resistance value.

17. The method of claim 16, wherein the first superhard material is distinct from the second superhard material, the first and second target materials are the same, and the first and second set of test conditions are the same.

18. The method of claim 17, further comprising:
determining that the first superhard material is advantageous over the second superhard material when the first efficiency ratio is higher.

19. The method of claim 16, wherein the first and second superhard material are identical and the first and second set of test conditions are different.

20. The method of claim 19, further comprising:
determining that the first and second superhard components perform better under the first set of test conditions than under the second set of test conditions when the first efficiency ratio is greater than the second efficiency ratio.
